(19) United States
(12) Patent Application Publication
(10) Pub. No.: US 2023/0293053 A1
(43) Pub. Date: Sep. 21, 2023

(54) IN VIVO SCREENING ARRAY

(71) Applicant: UNIVERSITY OF WASHINGTON, Seattle, WA (US)

(72) Inventors: Kelly Rose Stevens, Seattle, WA (US); Chelsea Fortin, Seattle, WA (US); Colleen O'Connor, Seattle, WA (US)

(73) Assignee: UNIVERSITY OF WASHINGTON, Seattle, WA (US)

(21) Appl. No.: 18/006,413

(22) PCT Filed: Aug. 12, 2021
(86) PCT No.: PCT/US2021/045813

§ 371 Date: Jan. 23, 2023

Related U.S. Application Data
(60) Provisional application No. 63/065,694, filed on Aug. 14, 2020.

(51) Int. Cl. A61B 5/145; A61B 5/00

(52) U.S. Cl. CPC A61B 5/14503; A61B 5/4244

(57) ABSTRACT

Systems, materials, and methods for making and using in vivo screening and highly parallel tissue grafting assay arrays are described. An implantable assay array may include a biocompatible substrate. The biocompatible substrate may define a number of mutually isolated microcompartments. The number of mutually isolated microcompartments may be determined based at least in part on a size of the implantable assay array. The size of the implantable assay array may be determined based at least in part on a size of an implantation subject.

### CROSS-REFERENCE TO RELATED APPLICATION

The present application claims the benefit of co-pending U.S. Provisional Patent Application No. 63/065,694, filed Aug. 14, 2020, entitled, “IN VIVO SCREENING ARRAY,” the disclosure of which is incorporated herein in its entirety.

### STATEMENT OF GOVERNMENT LICENSE RIGHTS

This invention was made with Government support under Grant No. HL137188 awarded by the National Institutes of Health. The Government has certain rights in the invention.

### BACKGROUND

The ability for tissue to regenerate is an ancient survival mechanism that has evolved in all multicellular animals. For example, first discovered over 2700 years ago, the mammalian liver has the remarkable capacity to fully regenerate when injured. It has been observed that when up to two-thirds of a normal liver is surgically resected, regenerative factors are released into the bloodstream and the liver can completely regenerate itself. To date, the mechanisms that drive the liver regeneration process remain elusive.

It is estimated that more than 120 million people suffer from chronic liver disease and cirrhosis globally. Of these, approximately 1.3 million people die annually. Conditions including alcoholism, nonalcoholic fatty liver disease, autoimmune hepatitis, hepatitis B virus, hepatitis C virus, hemochromatosis (iron buildup in the body), Wilson's disease (copper accumulation in the liver), infection (e.g., syphilis and brucellosis), prescription medications, and inherited disorders such as cystic fibrosis, lead to liver damage and scarring. Every time the liver is injured, whether by disease, alcohol consumption, or another cause, the liver tries to repair itself, forming scar tissue. This buildup of scar tissue, known as fibrosis, can lead to the deadly condition cirrhosis.

In cirrhosis, scar tissue essentially encases regenerating liver cells, or hepatocytes, in nodules, binding them and preventing regeneration. In humans, cirrhosis causes fatigue, appetite loss, nausea, swelling in the legs or abdomen, weight loss, jaundice, and/or hepatic encephalopathy (difficulty thinking, confusion, drowsiness, and slurred speech). Cirrhosis is responsible for over 60% of liver transplants. However, liver transplantation presents additional challenges such as size mismatches. Although 6,000 liver transplants occur annually, 1,500 adults and children die each year while awaiting a transplant. Aside from liver transplantation, there is no effective medical therapy for end-stage liver disease.

Further, the liver is a common site for cancer metastases and aggressive resections in malignancy. Treatments are often limited by hepatic functional reserve because surrounding liver tissues are too damaged to support cell grafting.

Much of the early knowledge about liver regeneration has resulted from studies in rodents. Such studies identified several key growth factors, cytokines, and signaling pathways. For example, in rodents, liver regeneration is orchestrated by diverse circulating factors, such as growth factors and cytokines, which bind key receptors on hepatocytes (e.g., MET and epidermal growth factor receptor [EGFR], interleukin-6 receptor [IL6R], tumor necrosis factor receptor 1 [TNFR], and farnesoid X receptor [FXR]). These pathways are thought to be functionally redundant, because elimination of no single receptor alone has been found to cause or stop rodent liver regeneration. Recently, one benchmark study demonstrated that liver regeneration could be abolished by eliminating two receptors simultaneously (MET and EGFR). However, the integrated roles of other major pathways (e.g., IL6, TNFR, FXR) is not known. Moreover, recent advances in highly parallel genomics have further implicated hundreds of new genes involved in the liver regeneration process. The degree to which pathways identified in rodent contribute to human liver regeneration is a critical yet experimentally intractable challenge. A critical hurdle is to shed light on how these integrated pathways drive human liver regeneration, and to leverage such pathways for therapeutics such as cell or tissue-based therapies.

Therefore, there exists a need for a high throughput tissue (e.g., liver) regeneration platform to understand the complex array of the microenvironmental factors involved in tissue (e.g., liver) regeneration and repair, the integrated role microenvironmental factors have within tissue (e.g., liver), and to enable previously inaccessible studies by introducing higher throughput in vivo screening systems, and to facilitate the discovery of new therapeutic approaches for tissue (e.g., liver) regeneration and repair.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Systems, materials, and methods for making and using in vivo screening and highly parallel tissue grafting assay arrays are described. An implantable assay array may include a biocompatible substrate. The biocompatible substrate may define a number of mutually isolated microcompartments. The number of mutually isolated microcompartments may be determined based at least in part on a size of the implantable assay array. The size of the implantable assay array may be determined based at least in part on a size of an implantation subject.

In some embodiments, the implantation subject is a mouse, and the biocompatible substrate defines from 2 to about 500 microcompartments. The implantation subject may be a rat, and the biocompatible substrate may define from 2 to about 50,000 microcompartments. The implantable assay array may further include cells or microtissue disposed in the microcompartments. The cells or microtissue may be or include hepatocytes, endothelial cells, stromal cells, or a combination thereof.

In some embodiments, a microcompartment of the microcompartments includes a hydrogel formulation. The hydrogel formulation may be or include gelatin methacryloyl (GelMA) having a weight percent of about 1% to about 50%, poly(ethylene glycol) diacrylate (PEGDA) having a weight percent of about 1% to about 20%, fibrin, collagen, Matrigel, and cell media, or combinations thereof. The hydrogel formulation may be crosslinked and may be characterized by a compressive modulus from about 75 Pa to about 10 kPa. The hydrogel formulation may include a nodule.

In some embodiments, a microcompartment of the microcompartments includes a biological scaffold matrix, the biological scaffold matrix comprising collagen, fibrin, decellularized extracellular matrix, silk fibroin, hyaluronic acid, hyaluronan, alginate, agarose, and methacrylated hyaluronic acid, or combinations thereof.

In some embodiments, the implantable assay array further includes a cover disposed on a lateral surface of the biocompatible substrate, the cover overlying the microcompartments and enclosing the crosslinked hydrogel within the microcompartment. The cover may transmit at least a portion of electromagnetic radiation generated by luciferase. The biocompatible substrate may include PEGDA, GelMA, collagen, fibrin, decellularized extracellular matrix, silk fibroin, hyaluronic acid, hyaluronan, alginate, agarose, and methacrylated hyaluronic acid, or combinations thereof.

In some embodiments, the implantable assay array further includes one or more apertures to permit suturing upon implantation into the implantation subject. The implantable assay array may further include further comprising a backing surface, wherein the biocompatible substrate is disposed on the backing surface. The biocompatible substrate may be substantially impermeable to cells, extracellular matrix, or biological molecules. Each microcompartment may define a biologically isolated environment. Each microcompartment may define an internal volume from about 0.1 μL to about 1000 μL.

A method of producing an implantable assay array, as described above, may include incrementally depositing and photo-curing a hydrogel substrate precursor using a stereolithographic assembly. The hydrogel substrate precursor may be or include a first hydrogel monomer, a photoblocker, a first photoinitiator, and cell media or biological buffer. Incrementally depositing and photo-curing the hydrogel substrate precursor may form a biocompatible hydrogel substrate including a number of mutually isolated microcompartments. The method may include disposing a hydrogel formulation, cells and/or microtissues, and/or cell media into a microcompartment of the microcompartments. The hydrogel formulation may be or include a second photoinitiator and a second hydrogel monomer. The method may also include photo-polymerizing the hydrogel formulation.

In some embodiments, the number of mutually isolated microcompartments is determined based at least in part on a size of the implantable assay array. The size of the implantable assay array may be determined based at least in part on a size of an implantation subject to receive the implantable assay array. the hydrogel formulation may be a first hydrogel formulation, the microcompartment may be a first microcompartment, and the method may further include disposing a second hydrogel formulation into a second microcompartment of the microcompartments. The second hydrogel formulation may be different from the first hydrogel formulation. The cells or microtissues may be or include hepatocytes, endothelial cells, stromal cells, or a combination thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of an example implantable assay array, implanted in an example implantation subject, in accordance with embodiments of the present disclosure.

FIG. 2A is a schematic diagram of an example implantable assay array including microcompartments and apertures, in accordance with embodiments of the present disclosure.

FIG. 2B is a composite diagram of an example environment performing a calcein/ethidium homodimer assay to assess cell viability, in accordance with embodiments of the present disclosure.

FIG. 3A is an image of an example implantable assay array, including a biocompatible substrate, microcompartments, and apertures, in accordance with embodiments of the present disclosure.

FIG. 3B is a schematic diagram of an elevation view of the example implantable assay array of FIG. 3A, in accordance with embodiments of the present disclosure.

FIG. 4 is a composite diagram of an example implantation subject with an overlay of in vivo imaging of the implanted assay array, showing albumin promoter-driven bioluminescence as a readout of cell viability and function, in accordance with embodiments of the present disclosure.

FIG. 5 is a block flow diagram of an example process for making an implantable assay array, in accordance with embodiments of the present disclosure.

FIG. 6A is an image of an example immune-stained and cleared tissue array containing human umbilical vein endothelial cells (HUVECs) and normal human dermal fibroblasts (NHDFs) suspended in various material inks, in accordance with embodiments of the present disclosure.

FIG. 6B is a bar graph describing percent vessel area for a number of example test conditions investigated using an implantable assay array, in accordance with embodiments of the present disclosure.

FIG. 7A is a composite diagram illustrating an example immune-stained and cleared tissue array containing HUVECs and NHDFs suspended in either 3%, 5%, 10% or 15% GelMA ink, after explant from a mouse, in accordance with embodiments of the present disclosure.

FIG. 7B is a bar graph illustrating percent vessel area for each condition described in FIG. 7A, in accordance with embodiments of the present disclosure.

FIG. 8A is a graph illustrating example bioluminescence signals as a proxy for hepatic function in various inks over eight days in vitro, in accordance with embodiments of the present disclosure.

FIG. 8B is a graph illustrating example bioluminescence of primary rat hepatocytes for each condition described in reference to FIG. 7A after eight days of implantation in a mouse, in accordance with embodiments of the present disclosure.

FIG. 9A is a graph illustrating example bioluminescence as a proxy for hepatic function in various inks after eight days in vivo, in accordance with embodiments of the present disclosure.

FIG. 9B is a composite image of albumin-driven bioluminescence from an example explanted assay array, in accordance with embodiments of the present disclosure.

FIG. 10A is a composite diagram illustrating an example explanted assay array containing human fetal hepatocyte organoids suspended in a screen of material ink, in accordance with embodiments of the present disclosure.

FIG. 10B is a graph illustrating quantified percent vessel area for each condition described in reference to FIG. 9A, in accordance with embodiments of the present disclosure.

### DETAILED DESCRIPTION

While illustrative embodiments have been illustrated and described, it will be appreciated that various changes can be made therein without departing from the spirit and scope of the invention.

Prior efforts to build artificial liver tissue incorporating components composed of structurally organized hepatocytes, endothelial cells, and fibroblasts have shown liver regeneration upon implantation. However, testing formulation conditions and other experimental factors on such a platform of designer tissue is limited because only a single artificial liver tissue can be implanted in an individual animal. Therefore, there exists a need for a high throughput tissue (e.g., liver) regeneration platform, also referred to as Highly Parallel Tissue Grafting (HPTG), to understand the complex array of the microenvironmental factors involved in tissue (e.g., liver) regeneration, the integrated role microenvironmental factors have within tissue (e.g., liver), and to facilitate the discovery of new therapeutic approaches for tissue (e.g., liver) regeneration and repair. To that end, while the embodiments described herein focus on liver regeneration and repair experiments, it is understood that the HPTG techniques described are broadly applicable to other assays, including but not limited to drug candidate screening and tissue engineering across diverse fields of biomedicine, such as cancer and cardiovascular disease.

To that end, in some embodiments an implantable HPTG platform permits investigation of a number of formulation and “seed” factors in parallel in a single implantation subject. HPTG can facilitate the use of a “combinatorial regeneration array,” also referred to as an implantable assay array, to test many different cell types, subtypes, or genetic permutations, tissue or extracellular microenvironment formulations, or other experimental factors, as well as replicates. In an illustrative example, HPTG permits a plurality of simultaneous in vivo experiments, also referred to as in vivo screening, to assess the integrated role of candidate factors in tissue (e.g., human or animal liver) regeneration, while also reducing the number of implantation subjects and the number of surgical procedures demanded.

In an illustrative example, an implantable assay array is to be implanted in a mouse for an HPTG trial. The implantable assay array includes a biocompatible substrate defining a number of mutually isolated microcompartments. The HPTG trial includes testing a number of conditions by loading each microcompartment of the implantable assay array with a hydrogel that includes cells or cell tissues, fibrin, collagen, Matrigel, and cell media. A subset of the microcompartments includes a biological scaffold matrix. The biological scaffold matrix includes collagen, fibrin, decellularized extracellular matrix, silk fibroin, hyaluronic acid, hyaluronan, alginate, agarose, and methacrylated hyaluronic acid. Each microcompartment includes cells from different humans or animals. A subset of the microcompartments include combinations of different types of cells, different subtypes of cells, cells from different genetic or epigenetic backgrounds, cells with one or more genetic permutations (e.g., knocked out genes), or other cells that may be selected for in vivo screening using the HPTG platform. The cells or cell tissues include hepatocytes, endothelial cells, and stromal cells.

Once prepared, the HPTG trial of the example includes implanting the implantable assay array in a mouse, in the region of the mouse's abdomen. During the period of engraftment in the host, cells within microcompartments of the implantable assay array generate luminescence emission in vivo, through the action of albumin-driven luciferase. In this way, various measures of cell viability and function are derived through measurement of transdermal in vivo emission. After the completion of the implantation period, the implantable assay array is explanted and processed for further analysis, including structural, chemical, and genetic parameters, such as vascularization or protein expression. Throughout the implantation period, each microcompartment is maintained as an isolated environment by covering the implantable assay array with a biocompatible cover that seals the microcompartments or via apposition, covering, and surgical suturing to a host tissue or fat pad (e.g., gonadal or mesenteric fat), and by the biocompatible substrate that is substantially impermeable to cells, extracellular matrix, and biological molecules.

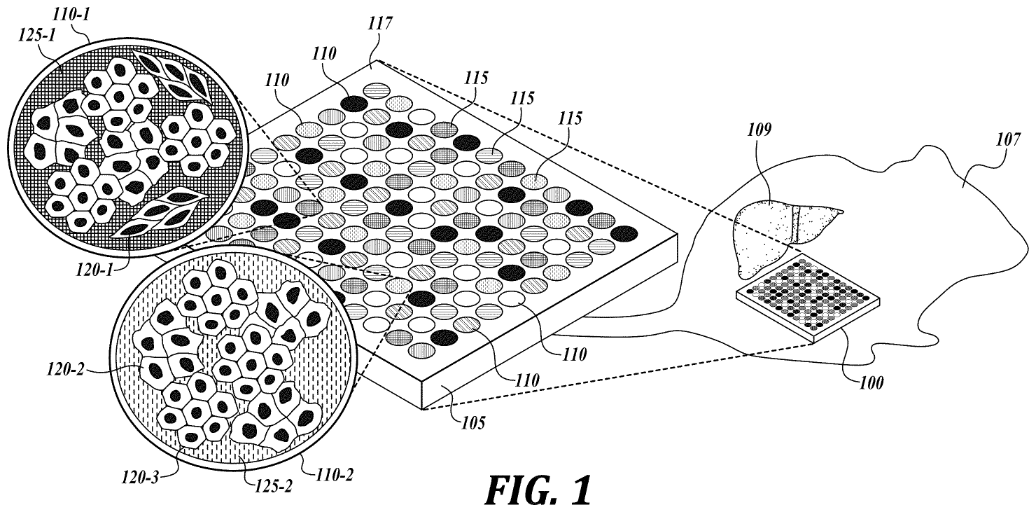

FIG. 1, is a schematic diagram of an example implantable assay array 100, implanted in an example implantation subject 107, in accordance with embodiments of the present disclosure. The example array 100 includes a biocompatible substrate 105 that defines a number of microcompartments 110. The microcompartments 110 are mutually isolated, to each define an isolated biological environment 115. The biological environment 115 of each microcompartment 110 includes one or more types of cells 120 in a hydrogel formulation 125. In the context of the example array 100, the term “mutually isolated” refers to each microcompartment 110 maintaining a biochemical configuration that may differ between the microcompartments 110, thereby facilitating parallel and combinatorial assays, as in HPTG trials.

In some embodiments, the biocompatible substrate 105 comprises a lateral surface 117 and each mutually isolated microcompartment 110 is defined by an opening in the lateral surface 117 (e.g., forming a depression or well in the lateral surface 117). The biocompatible substrate 105 includes at least 2 microcompartments. The number of mutually isolated microcompartments 110 may be determined based at least in part on a size of the array 100 and/or by the size of the individual microcompartments 110 (e.g., wells or holes). For example, each microcompartment 110 may define an internal volume from about 0.1 μL to about 1000 μL. The size of the array, in turn, may be determined based at least in part on a size of an implantation subject 107. For example, where the implantation subject 107 is a mouse, the biocompatible hydrogel substrate may define from about 2 to about 500 or more microcompartments 110. In another example, where the implantation subject 107 is a rat, the biocompatible hydrogel substrate may define from about 2 to about 50,000 or more microcompartments 110. It is understood that the implantation subject 107 may be an animal other than a mouse or a rat. In some embodiments, the implantation subject 107 is a metazoan animal, such as, for example, a reptile, a bird, or a mammal. The implantation subject 107 may be a human, a non-human primate, a rodent (such as mouse, rat, guinea pig, and the like), a dog, a cat, a sheep, a horse, a cow, a pig, a goat, or other metazoan animal, as would be understood by a person having ordinary skill in the art. In some embodiments, the implantation subject 107 is a transgenic, chimeric, and/or humanized animal model. For example, the implantation subject 107 may be a FRGN mouse, where “FRG” refers to a [Fah(—/—)Rag2(—/—)Il2rg(—/—)] mouse, as would be understood by a person having ordinary skill in the art, that may have an injured mouse liver 109 or liver that is repopulated with primary human hepatocytes to include a humanized liver or repopulated with other human cells to include another or a different humanized organ. In some embodiments, the implantation subject is an immunodeficient mouse that is reconstituted with a human immune compartment, for example, by reconstitution with mature lymphocytes from a human donor.

While it may be advantageous to include as many microcompartments 110 as possible within the implantation subject 107 by reducing the volume of each microcompartment 110, thereby providing a larger number of testable experimental conditions, the volume of the microcompartments 110 may be limited at least in part by the type of experiment to be conducted, the size of the cells or tissues being investigated, or other factors of the experimental system other than the size of the implantation subject 107. In this way, the number of microcompartments 110 may be determined within a range, based at least in part on the size of the implantation subject 107, where larger implantation subjects permit larger numbers of microcompartments 110 to be defined in the biocompatible substrate 105. In some embodiments, the biocompatible substrate 105 defines from at least 2 to about 5,000 microcompartments 110 or any range therein, such as 2 to about 4000, 2 to about 3000, 2 to about 1000, 2 to about 750, 2 to about 500, 2 to about 400, 2 to about 300, 2 to about 200, 2 to about 100, 2 to about 75, 2 to about 50, 2 to about 25, 25 to about 400, 25 to about 300, 25 to about 200, 25 to about 100, 25 to about 75, 25 to about 50, 50 to about 400, 50 to about 300, 50 to about 200, 50 to about 100, 50 to about 75, or the like.

The biocompatible substrate 105 may be or include a biocompatible material that is substantially impermeable, semi-permeable, or permeable, to cells, extracellular matrix, and/or biological molecules. In this context, the term “biocompatible” refers to a material that does not interrupt, damage, denature, destroy, or otherwise inhibit the viability of the implantation subject 107 or the cells 120 seeded in the microcompartments 110, as would be understood by a person having ordinary skill in the art. In this context, the term “biological molecules” refers to proteins, lipids, carbohydrates, nucleic acids, viruses, and/or small molecules with biological targets, as would be understood by a person having ordinary skill in the art. In this context, the term “substantially” refers to the biocompatible substrate 105 acting as a physical or chemical barrier to species transport between individual microcompartments 110 that would influence the development of the cells 120 contained therein. In this context, the term “semi-permeable” refers to the biocompatible substrate 105 being permeable to molecular species, while acting as a physical barrier to cell migration into the material of the biocompatible substrate 105. For example, the biocompatible substrate 105 may permit transport of water molecules and/or small ions (e.g., sodium, potassium, chloride, or the like) between microcompartments 110, but may significantly limit transport of cells, extracellular matrix, and/or biological molecules. In another example, the biocompatible substrate may permit transport of water molecules, small ions, larger species (e.g., proteins), but may significantly limit transport of cells. Advantageously, partial impermeability and/or substantial impermeability permits the biocompatible substrate 105 to define multiple isolated environments 115, thereby facilitating experimental investigation of different conditions or parameters, including but not limited to tissue grafting, tissue vascularization optimization and modeling, drug candidate screening, cellular grafting, biomaterial or hydrogel optimization for in vivo applications, disease modeling, genetic “hit” validation from in vitro screens, bioink screening, and the like. The term “bioink” is described in more detail in reference to Examples 1-4.

In some embodiments, the biocompatible substrate 105 may be or include a hydrogel, formed from crosslinked (e.g., by photo-polymerization) poly(ethylene glycol) diacrylate (PEGDA), gelatin methacryloyl (GelMA), or a combination thereof. In some embodiments, the biocompatible substrate 105 is formed of poly(ethylene glycol) diacrylate (PEGDA) without including GelMa. In some embodiments, the biocompatible substrate 105 includes a photoinitiator, a photoblocker or a dye, and cell media or buffer. The cell media or buffer can be or include media that is appropriate to facilitate cell culture and viability, based at least in part on the cells 120 or tissue that are included in the array. In some embodiments, the photoinitiator is lithium phenyl-2,4,6-trimethylbenzoylphosphinate (LAP), the photoblocker is tartrazine. In an illustrative example, the biocompatible substrate 105 includes: 3.25:10 weight % 3.4 kDa PEGDA:GelMA, 1.591 mM tartrazine (photoblocker), 17 mM LAP Lithium phenyl-2,4,6-trimethylbenzoylphosphinate (photoinitiator), and PBS (phosphate buffered saline).

The material composition of the biocompatible substrate 105 is understood to influence bulk and surface material properties with which the contents of each microcompartment 110 interact. For example, the compressive modulus, which may serve to simulate disease state of an organ (e.g., the liver), may be modulated at least in part by the relative composition of PEGDA to GelMA in the hydrogel formulation. In some embodiments, a higher compressive modulus simulates a more diseased organ. As such, the biocompatible substrate 105, as well as each isolated biological environment 115, may be formed to be characterized by a compressive modulus between about 75 Pa and about 10 kPa. Accordingly, in some embodiments, the implantable assay array 100 and isolated biological environments 115 are is characterized by compressive moduli that varies among the mutually isolated microcompartments 110 from about 75 Pa to about 10 kPa or any range therein. In this context, the term “about” is used to indicate a value within 10% of the stated value.

As described in more detail below, a hydrogel formulation 125 can be added to the microcompartments 110 to simulate multiple different environments 115 across the example array 100. In some embodiments, the microcompartments 110 or a subset of the microcompartments 110 are provided with cell media, without a hydrogel monomer. In an illustrative example, a microcompartment 110 may be seeded with cells 120 in cell media that over time will remodel and secrete extracellular matrix, referred to as a “cell-only” tissue.

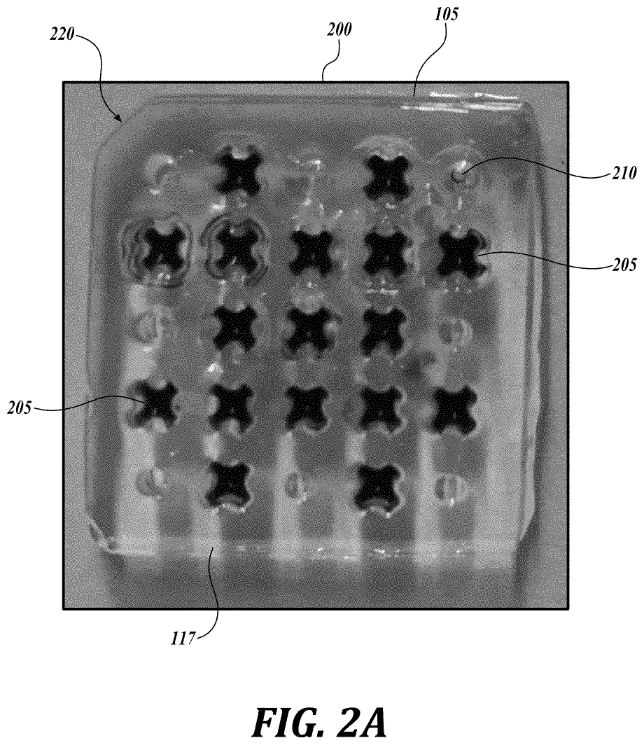

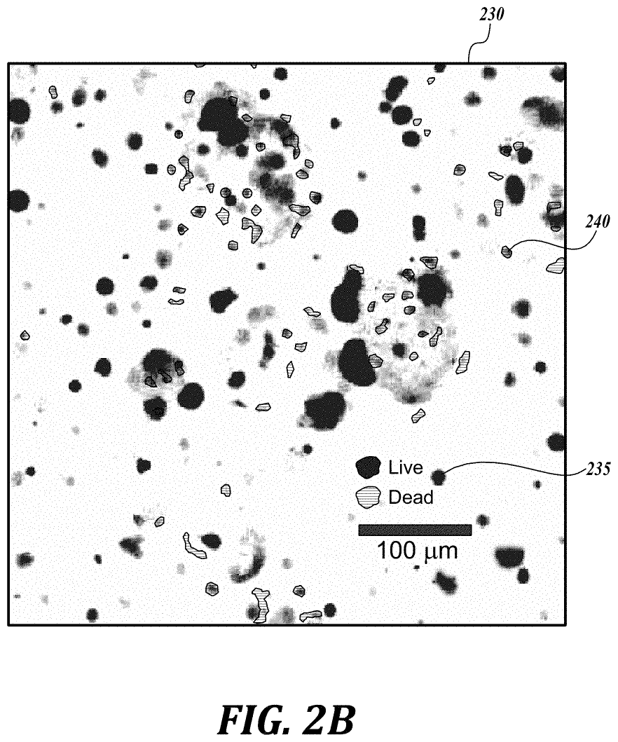

The mutually isolated microcompartments 110 are configured into any shape that is capable of receiving cells or tissue seeds for culture, along with optional supplementation with media, support cells, and other experimental co-factors, as desired. For example, the plurality of mutually isolated microcompartments 110 can be cylindrical, polyhedral, cube-shaped, or other shapes (e.g., cross-shaped, as in FIG. 2A). For example, each microcompartment 110 can define an approximately cylindrical volume with a diameter from about 1 mm to about 3 mm, with a height of between about 0.5 mm to about 3 mm. In this context, the term “about” is used to indicate a value within 10% of the stated value.

The example array 100 may be substantially planar, but may also assume alternative form factors. Advantageously, planar configurations improve accessibility of the mutually isolated microcompartments 110, improve compatibility with additive manufacturing techniques for soft materials, and reduce material consumption. For example, the example array 100 may be configured as a rectangular prism, with similar proportions as a multi-well plate, as illustrated in FIG. 1. In some embodiments, the example array 100 is shaped, as during additive manufacturing, to a form or shape of a specific implantation location of the implantation subject. For example, the example array 100 may be formed such that it conformably couples with a surface, including but not limited to a surface of the heart or other internal tissue.

In an illustrative example, where the implantation subject 107 is a mouse, the biocompatible substrate 105 may be about 5 mm long, about 5 mm wide, and about 1 mm high. In larger subjects, by contrast, the biocompatible substrate 105 may have a size up to or about 500 mm long, about 500 mm wide, and about 100 mm high, or any range therein. For example, where the implantation subject 107 is a rat, characterized by a relatively larger abdominal region than a mouse, the size of the biocompatible substrate 105 may be larger than for a mouse and may include a larger number of similarly sized microcompartments 110. For example, for a rat, the example array 100 may have a size up to or about 80 mm width and about 80 mm length of the lateral surface 117, with depth similar or exceeding that for a mouse. In contrast, where the implantation subject 107 is a pig, the example array 100 may have a size of up to or about 300 mm width and 300 mm length at the lateral surface 117. It is understood that intraspecies variability in size, such as that due to dimorphism, breed traits, or the like, may influence the size of the array 100.

In some embodiments, the array 100 is manufactured and seeded with cells 120 as part of a manufacturing process, prior to being provided to an end user, such as a scientist, laboratory technician, or the like. As such, the array 100 may be prepared for a species or breed of implantation subject 107 rather than for an individual animal. In this way, a number of standard sizes may be prepared, based at least in part on the average size of the species of the implantation subject 107. In turn, the number of microcompartments 110 may be determined within a window based on the volume of the microcompartments 110 to be used for a particular assay.

In some embodiments, multiple different microenvironments 115 are defined in the array 100, through modifying the types of cells 120, subtypes of cells 120, genetic permutations to cells 120, cell donor or individual patient(s), hydrogel formulations 125, and/or other constituent materials that are disposed in each respective microcompartment 110. The cells and/or microtissue 120 may be derived from one or more sources, including, but not limited to a metazoan animal, such as, for example, a reptile; a bird; and a mammal such as human, non-human primate, rodent (such as mouse, rat, guinea pig, or the like), dog, cat, sheep, horse, cow, pig, goat, or the like.

In an illustrative example, a first microcompartment 110-1 of the mutually isolated microcompartments 110 is seeded with first cells and/or microtissues 120-1, second cells and/or microtissues 120-2, and third cells and/or microtissues 120-3 in a first hydrogel formulation 125-1. In contrast, a second microcompartment 110-2 may include the second cells and/or microtissues 120-2 and the third cells and/or microtissues 120-3 in a second hydrogel formulation 125-2, where the number of microcompartments 110 may be determined at least in part by the number of formulations or combinations of environments 115 to be investigated. The cells and/or microtissues 120 can be or include cells intended for implantation. For example, the cell or microtissue can be or include endothelial cells, endothelial cells from human umbilical vein, endothelial cells from adult and/or fetal liver, endothelial cells from fetal lung, endothelial cells from induced pluripotent stem cells, induced pluripotent stem cell-derived cell populations, fibroblasts, hepatocytes, hepatic fetal organoids, hepatoblast organoids, stromal cells of one or more types, such as fibroblasts, pericytes, or smooth muscle cells, or combinations thereof. In turn, the first hydrogel formulation 125-1 and the second hydrogel formulation 125-2, as well as subsequent formulations, may exhibit different material properties configured to simulate different disease states of an organ, and/or may include cells from different human donors or different candidate drugs as part of a drug screening assay. Microcompartment formulations might also include various biological molecules or delivery or controlled release vehicles, such as nanoparticles, carbon nanotubes, and the like.

One or more of the cells and/or microtissues 120 and at least one experimental co-factor can be introduced into the microcompartments 110 as a hydrogel formulation 125. In some embodiments, each of the plurality of mutually isolated microcompartments 110 includes a respective hydrogel formulation 125. The hydrogel formulation 125 of each of the plurality of mutually isolated microcompartments may be the same or different. In some embodiments, the experimental co-factor is the hydrogel formulation 125, an extracellular matrix factor, a cell type, and/or a cell population. The array 100 is not limited to two environments 115, but rather may include a number of environments 115 equal to the number of compartments 110. Even so, the number of environments 115 may be fewer than the number of compartments 110 where at least a subset of the compartments 110 include replicated environments.

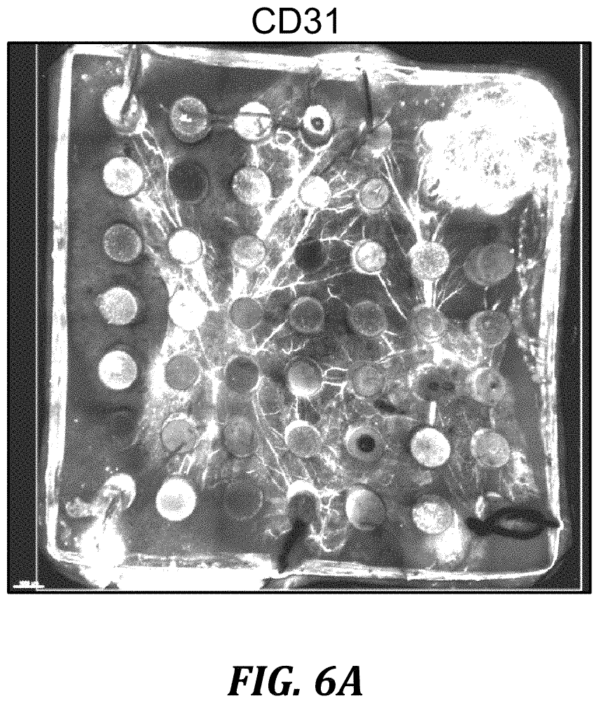

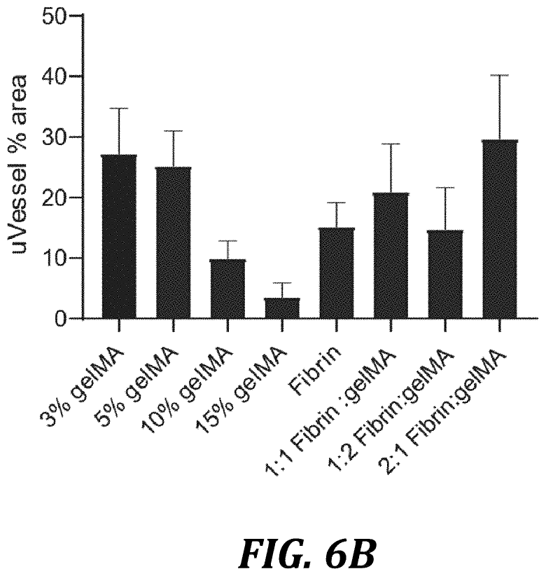

In some embodiments, the hydrogel formulation 125 of each of the plurality of mutually isolated microcompartments independently comprises a GelMA formulation having a weight percent of about 3% to about 20%, a PEGDA formulation having a weight percent of about 1% to about 20%, fibrin, collagen, Matrigel, and media, or combinations thereof. Illustrative examples of hydrogel formulations 125 are described in detail in reference to FIGS. 6A-10B

In some embodiments, the hydrogel formulation 125 comprises a biological scaffold matrix. For example, the biological scaffold matrix can comprise collagen, fibrin, decellularized extracellular matrix, silk fibroin, hyaluronic acid, hyaluronan, alginate, agarose, methacrylated hyaluronic acid, and the like, or any combination thereof. In some embodiments, the hydrogel formulation 125 comprises PEGDA, GelMA, collagen, fibrin, decellularized extracellular matrix, silk fibroin, hyaluronic acid, hyaluronan, alginate, agarose, methacrylated hyaluronic acid, or any combination thereof.

As part of simulating diverse biological environments, the hydrogel formulation 125 may be patterned during fabrication. Patterning may include spatially localized crosslinking of hydrogel monomers to form one or more nodules within a microcompartment 110. The term “nodule” in this context refers to a region of nonuniform material properties, including but not limited to compressive modulus. In this way, the environment 115 formed may include a metastructure simulating the form of an organ, tissue, or other environment. In an illustrative example, an environment 115 prepared to simulate a human liver may include multiple nodules of higher stiffness within a matrix of relatively lower stiffness. The higher stiffness nodules may be formed by prolonged or higher intensity irradiation of a localized region of the hydrogel formulation 125 by targeted near-UV photons, causing an interaction volume to harden near the region.

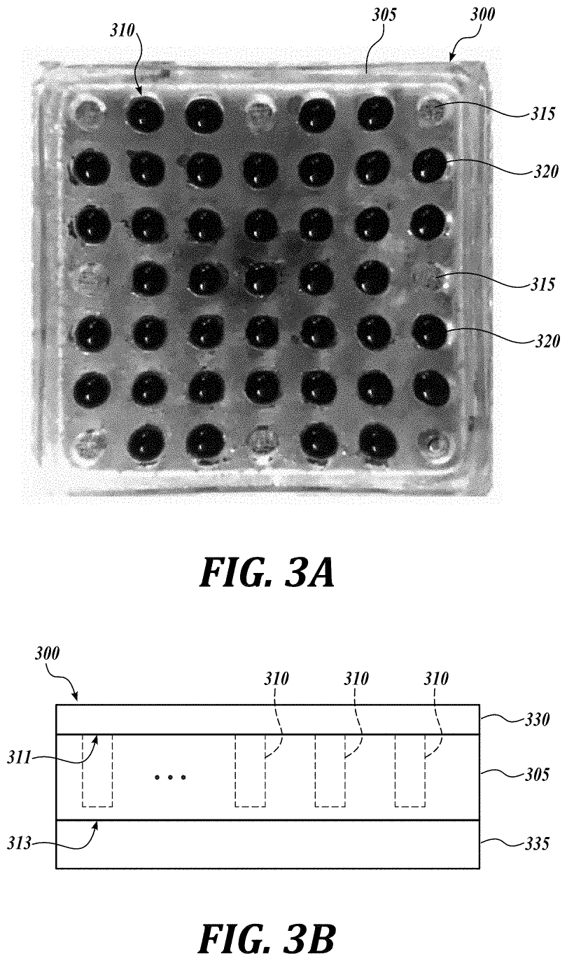

FIG. 2A is a schematic diagram of an example implantable assay array 200 including microcompartments 205 and apertures 210, in accordance with embodiments of the present disclosure. The example array 200 is an example of the implantable assay array 100 of FIG. 1, prepared for implantation in an implantation subject that is an example of the implantation subject 107 of FIG. 1. The example array 200 includes a number of microcompartments 205 that is determined based at least in part on a size of the implantation subject. For the example array 200, the implantation subject is a humanized mouse (e.g., a FNGR mouse), and includes seventeen microcompartments 205. To facilitate implantation, the example array 200 includes eight apertures 210, positioned around the periphery of the microcompartments 205, which are through-holes, through the biocompatible substrate 105. The apertures 210 permit the example array 200 to be implanted into the implantation subject, for example, onto the perigonadal fat pad of the mouse. While the apertures 210 are illustrated as being spaced regularly around the periphery, the number of apertures 210 may be consistent for different numbers of microcompartments 205. For example, while the example array 200 includes eight apertures 210 for seventeen microcompartments 205, another array may include eight apertures 210 for forty-one microcompartments 205, as described in more detail in reference to FIG. 3A.

The microcompartments 205 of example array 200 are characterized by an effective diameter of about 2 mm. In this context, the term “about” refers to a value within 10% of the stated value. The microcompartments 205 are illustrated having a cruciform cross-section, such that the effective diameter may measure the widest width of the microcompartments 205. In some embodiments, the microcompartments 205 are separated by a distance equivalent to the effective diameter or greater. As such, the overall dimensions of the example array 200 may be from about 20 mm to 28 mm, square, on the lateral surface 117, and from about 0.5 mm to about 3 mm in depth (into the plane of FIG. 2A). The cited dimensions are illustrative and are not intended to be limiting. Instead, the dimensions of the example array 200 may be determined at least in part by the size of the implantation subject, the average size of the type of implantation subject, the volume of the microcontainers 205, or a combination thereof.

As described in reference to FIG. 1, each microcompartment 205 may define a volume that is determined at least in part by the type of environment 115 being provided. For example, some experimental environments 115 may include larger volumes of cells 120 and/or hydrogel formulations 125. In this way, it is understood that the example array 200 illustrates a non-limiting example of the dimensions of the microcompartments 205. In an illustrative example, example array 200 is seeded with seventeen distinct human artificial liver cell or microtissue 120 samples, representing different combinations of human hepatocyte, endothelial cells, and fibroblasts (e.g., first cells 120-1, second cells 120-2, and third cells 120-3) in hydrogels with varying material properties (e.g., compressive modulus), thereby defining seventeen different environments 115 cellularized or not cellularized that are mutually isolated from each other.

In some embodiments, the example array 200 comprises a registration marking 220 to facilitate orientation and reliable identification of individual microcompartments 205 in reference to the registration marking 220. As illustrated, the registration marking 220 is a transected corner of the example array 200. In some embodiments, the registration marking 220 may be or include one or more differently shaped microcompartments 205 or apertures 210 and/or a feature of the biocompatible substrate 105, such as a molded feature formed in the biocompatible substrate 105.

FIG. 2B is a composite diagram of an example environment 230 performing a calcein/ethidium homodimer assay to assess cell viability, in accordance with embodiments of the present disclosure. The example environment 230 is an example of an environment 115 of FIG. 1. FIG. 2B, therefore, illustrates a microscope image of a hydrogel (e.g., hydrogel formulation 125 of FIG. 1) including live cells 235 and dead cells 240.

In the example environment 230, a microcompartment 110 of the example array 110 is seeded with human hepatocytes, along with hydrogel, cell medium, and/or other components as described in more detail in reference to FIG. 1. As would be understood by a person having ordinary skill in the art, a calcein/ethidium homodimer assay is a dual-fluorophore cell viability assay. It uses two probes, calcein AM and ethidium homodimer (EthD-1), to detect live and dead cells simultaneously through fluorescence at different wavelengths. While the assay uses visible wavelengths registered as colors (e.g., red and green light), live cells 235 are shown as solid regions and dead cells 240 are shown as patterned regions on a white background. As illustrated, the sampled environment 230 includes live cells 235. Consequently, encapsulation of the human hepatocytes within the microcompartments 110 of the example array 100 is understood to preserve, maintain, and/or have minimal or no detectable effects on cellular viability. It is also understood that the example environment 230, and thus the implantable assay array 100, and constituent components, are biocompatible.

FIG. 3A is an image of an example implantable assay array 300, including a biocompatible substrate 305, microcompartments 310, and apertures 315, in accordance with embodiments of the present disclosure. The example array 300 is an example of implantable assay array 100 of FIG. 1. As described in reference to FIG. 2A, example array 300 illustrates an HPTG array for implantation in a mouse, including 41 microcompartments 310 and eight apertures 315. The number of microcompartments 310, illustrated as cylindrical volumes, is determined at least in part by the size of the implantation subject, for example, based at least in part on the area of the perigonadal fat pad of the mouse.

The example array 300 may be prepared to include multiple environments 320 such that the stiffness of the microtissues within the example array 300 mimic the range of conditions corresponding to heterogeneous liver tissue. To accomplish the variable stiffness, the example array 300 may include 41 individually addressable microcompartments 310, which may be examples of the microcompartments 110 of FIG. 1. In some embodiments, a hydrogel formulation added to the microcompartments 310 is varied by modifying a composition of the hydrogel constituent materials (e.g., percent weight-to-volume of GelMA relative to PEGDA) to alter crosslinking density, once the hydrogel formulation is polymerized and/or crosslinked. In this way, hydrogels may be prepared across a range of values of compressive modulus. In an illustrative example, the environments 320 may be prepared according to different values of compressive modulus, including but not limited to about 25 Pa or more, about 50 Pa or more, about 75 Pa or more, about 100 Pa or more, about 250 Pa or more, about 500 Pa or more, about 1 kPa or more, about 3 kPa or more, about 6 kPa or more, and about 10 kPa or more. It is understood that the number of values is not limiting and may be larger where the range of test conditions is larger. Similarly, the cited values are intended as non-limiting examples describing inclusive open-ended ranges. For example, where the environment 320 is prepared to simulate a tissue that exhibits stiffness above 10 kPa, the hydrogel may be formed with a corresponding compressive modulus, thereby permitting the simulation of additional and/or alternative physiological environments as part of HPTG, drug screening, or other assays. Advantageously, the environments 320 may be prepared to mimic a range from sub-physiologic to pathological (e.g., cirrhotic) stiffness of the extracellular matrix of an organ (e.g., a human liver).

As described in more detail in reference to FIG. 2A, the example array 300 may be fabricated such that each environment 320 (corresponding to different extracellular matrix conditions) is replicated. In an illustrative example of eight conditions, corresponding to values of about 75 Pa, about 100 Pa, about 250 Pa, about 500 Pa, about 1 kPa, about 3 kPa, about 6 kPa, and about 10 kPa, the example array 300 includes five microcompartments 310 replicating each respective environment 320. In some embodiments, compressive stimulus values may be quantified by atomic force microscopy or other assays. Matrix pore size and crosslinking density may be analyzed by scanning electron microscopy to validate any confounding variables.

FIG. 3B is a schematic diagram of an elevation view of the example implantable assay array 300 of FIG. 3A, in accordance with embodiments of the present disclosure. The example array 300 includes the biocompatible substrate 305, a number of microcompartments 310, a cover 330, and a backing surface 335. The structures illustrated as part of FIG. 3B may be examples of structures included as part of the example array 100 of FIG. 1.

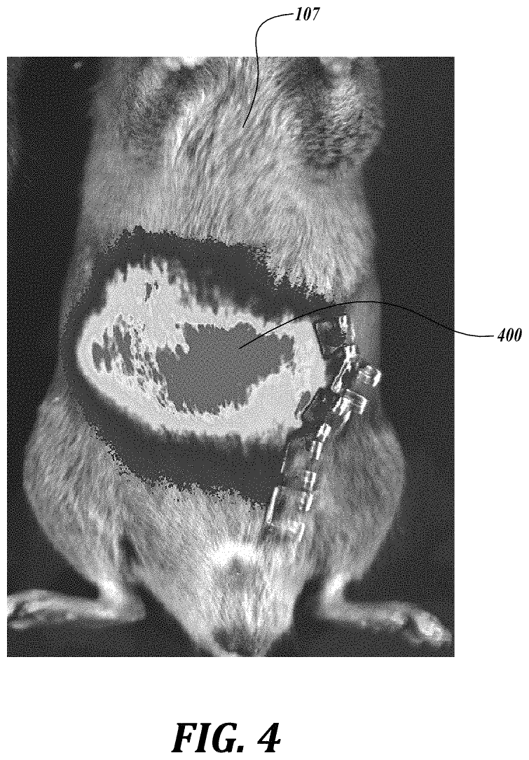

In some embodiments, the biocompatible substrate 305 comprises a cover 330 disposed over the lateral surface (e.g., lateral surface 117 of FIG. 1) of the biocompatible substrate 305. In this way, the cover 330 may cover the mutually isolated microcompartments 310. Where the biocompatible substrate 305 defines the compartments 310 as closed ended “wells,” the cover 330 can serve to seal the mutually isolated microcompartments 310, for example, subsequent filling with the desired contents. In some embodiments, the cover 330 includes a second hydrogel formulation that is distinct from that of the biocompatible substrate 305. Further, as described in more detail in reference to FIG. 4, the cover 330 may be transmit at least a portion of electromagnetic radiation generated by luciferase. For example albumin-driven luciferase, such that the viability and proliferation of cells (e.g., cells and/or microtissue 120 of FIG. 1) may be monitored in vivo or after ex-plantation of the example array 300.

In some embodiments, the example array 300 includes the backing surface 335, such that the biocompatible substrate 305 is disposed on the backing surface 335. In some embodiments, the example array 300 includes both the backing surface 335 and the biocompatible substrate 305 in a unitary array defined by different material properties. For example, the backing surface 335 may be characterized by relatively higher stiffness than the biocompatible substrate 305, lower permeability than the biocompatible substrate 305, or other features. Further, the biocompatible substrate 305 and the backing surface 335 may include apertures 315 extending continuously through both the biocompatible substrate 305 and the backing surface 335. In this way, the backing surface 335 may provide additional structure for ease of handling and to provide durability during implantation and ex-plantation. The backing surface 335 can also serve to prevent infiltration of host tissue through one side of the microcompartment, so as to expose the microcompartments to host tissue only on the lateral surface 117.

While the microcompartments 310 are illustrated as terminating within the biocompatible substrate 305, in some embodiments, the microcompartments 310, defined by the biocompatible substrate, may define a first end 313 and a second end 311, such that the backing surface 335 encloses the microcompartments 310 at the first end 313 and the cover 330 encloses the microcompartments 310 at the second end 311. Alternatively, the second end 311 may be enclosed by a substantially impermeable internal structure of the implantation subject 107 (e.g., a fat pad). As with the biocompatible substrate 305, the cover 330 and the backing surface 335 may include biocompatible materials, including but not limited to crosslinked hydrogels. In some embodiments, the backing surface 335 is a solid material. Additionally and/or alternatively, the backing surface 335 may be a porous material, or other material exhibiting relatively higher stiffness or other material properties permitting the biocompatible substrate 305 to be supported.

FIG. 4 is a composite diagram of an example implantation subject 107 with an overlay of in vivo imaging of the implanted assay array 100, showing albumin promoter-driven bioluminescence 400 as a readout of cell viability and function, in accordance with embodiments of the present disclosure. The bioluminescence 400 is illustrated as an example of the luciferase in vivo monitoring described in reference to FIG. 1 and FIG. 3B. The implantation subject 107 is shown as a mouse, but it is understood that, in some embodiments, the implantation subject 107 is another type of animal.

In some embodiments, cells 120 (e.g., hepatocytes) are transduced with a lentiviral vector in which luciferase is expressed under an albumin promoter. In an illustrative example, at least a subset of the microcompartments 110 in the example array 100 are seeded with multiple cells and/or microtissues 120 (e.g., transduced hepatocytes, endothelial cells and fibroblasts) in a GelMa hydrogel 125. Cell viability, phenotype, and function may be confirmed in the microcompartments 110 using calcein/ethidium homodimer live imaging, bioluminescence imaging for spatially-localized albumin reporter activity, immunostaining for hepatic markers in clarified or sectioned tissue arrays, and metrics for secreted cell 120 (e.g., hepatocyte) products.

As illustrated, the bioluminescence 400 (e.g., luminescence emission generated by the transduced luciferase) may be monitored trans-dermally while the implantation subject 107 is alive (e.g., in vivo). Additionally and/or alternatively, the bioluminescence 400 may be monitored subsequent explantation of the example array 100, for example, following the period of time allotted for the experiments. Exemplary embodiments reporting transdermal luminescence measurement are reported in reference to the examples, below.

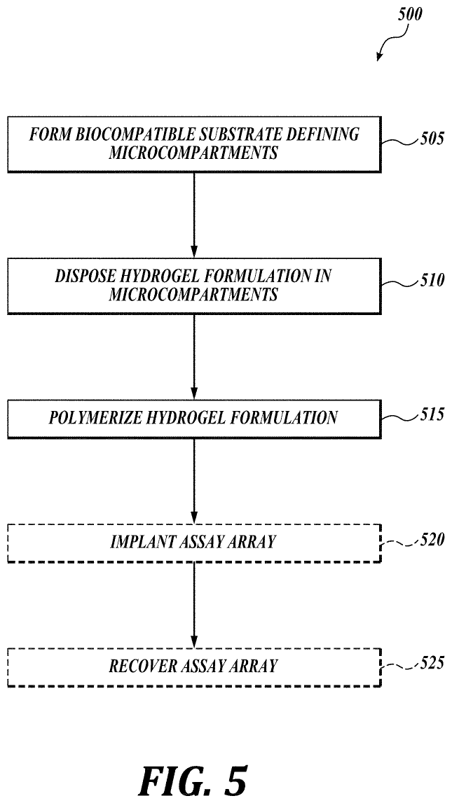

FIG. 5 is a block flow diagram of an example process 500 for making an implantable assay array, in accordance with embodiments of the present disclosure. The constituent operations of the example process 500 may be implemented in whole or in part by a computing device, for example, as part of an additive manufacturing assembly controlled automatically (e.g., without human intervention) by a computer controller. In some embodiments, the operations of example process 500 permit the preparation of various sizes and configurations of the example array 100, for example, based at least in part on the size of the implantation subject 107 identified to host the example array 100.

At operation 505, the example process 500 includes preparing the biocompatible substrate 105 by mixing a first hydrogel formulation, optionally comprising PEGDA and/or GelMA, with a photoblocker, a photoinitiator, and cell media. In some embodiments, operation 505 includes incrementally forming the biocompatible substrate 105 by additive manufacturing. For example, operation 500 may include printing the example array 100 using stereolithographic layer-by-layer assembly (e.g., SLA) in a device configured to print soft materials and/or other biocompatible materials, such as hydrogels. As described in more detail in reference to FIG. 1, the dimensions of the biocompatible substrate 105 and the number of the microcompartments 110 may be determined based at least in part on the size of the implantation subject 107. In this way, operation 505 may optionally include identifying an animal type corresponding to the implantation subject 107, for example, from a list of available implantation subjects for which information is available and accessing dimension information from a database storing the dimension information, as part of an automated approach to manufacturing implantable assay arrays in a separate physical location to the location of the implantation subject 107.

In some embodiments, preparing the biocompatible substrate 105 may optionally include washing the biocompatible substrate 105. For example, the biocompatible substrate 105 may be washed (e.g., in cell media) for a period of time (e.g., from about one day to about five days) to remove excess and unreacted monomers, photoinitiator, or other chemicals. It is understood that the period of time may be shorter than about one day or longer than about five day, based at least in part on material properties of the material from which the biocompatible substrate is formed, the transport properties of the various chemicals being removed, or other characteristics.

Subsequent to forming the biocompatible substrate 105, a second hydrogel formulation 125 may be prepared by mixing the photoinitiator, GelMA, cells and/or microtissue 120, and cell media, at operation 510. As described in more detail in reference to FIG. 1, operation 510 may include preparing multiple second hydrogel formulations 125 that are distinct, thereby permitting the example array 100 to serve as a comparative assay including multiple different environments 115. Operation 510 further includes disposing the second hydrogel formulation(s) 125 in the microcompartments 110 of the example implantable assay array 100. In some embodiments, the second hydrogel formulation 125 does not include GelMA or PEGDA, but rather includes cells and/or microtissue 120 and cell media, referred to as a “cell only” tissues, albeit including other components save hydrogels. A person having ordinary skill in the art would understand that various techniques are compatible with disposing the second hydrogel formulation(s) 125 into the microcompartments 110. For example, techniques may include, but are not limited to, manual pipetting, multi-pipetting, auto-pipetting, as well as other techniques suited to large scale manufacture as may be used in bio-manufacturing processes.

Subsequent to disposing second hydrogel formulation(s) 125 in the microcompartments 110, the example process 500 includes exposing the implantable assay array to electromagnetic radiation at operation 515. Operation 515 may include exposing the microcompartments individually, collectively, or locally (e.g., as part of patterning individual environments 115 to include nodules) to ultraviolet, near ultraviolet, or other wavelengths of electromagnetic radiation as photons, but may also include microwave radiation, thermal energy, or other crosslinking techniques that are compatible with viability of the cells and/or microtissues 120 and provide the intended material properties (e.g., compressive modulus) of the second hydrogel formulation(s) 125.

In some embodiments, exposure to the cross-linking energy may continue for a period of time sufficient to effect the intended chemical change without damaging the contents of the second hydrogel formulation(s) 125. For example, exposure duration may include from about 10 seconds to about 200 seconds, or any range therein, including but not limited to about 10 seconds or more, about 20 seconds or more, about 30 seconds or more, about 40 seconds or more, about 50 seconds or more, about 60 seconds or more, about 70 seconds or more, about 80 seconds or more, about 90 seconds or more, about 100 seconds or more, about 110 seconds or more, about 120 seconds or more, about 130 seconds or more, about 140 seconds or more, about 150 seconds or more, about 160 seconds or more, about 170 seconds or more, about 180 seconds or more, about 190 seconds or more, about 200 seconds, or more, including interpolations thereof.

In some embodiments, the example process 500 includes assembling the experimental assay array for HPTG assays within an implantation subject 107. In this way, the biocompatible substrate 105 may be disposed on a backing surface (e.g., backing surface 335 of FIG. 3B) or formed with a backing surface as a single piece with local material properties. Additionally or alternatively, the biocompatible substrate 105 may be covered (e.g., using cover 330 of FIG. 3B).

In some embodiments, creating the unique experimental microenvironments 110 includes adding one or more cells and/or microtissue 120, wherein each of the plurality of mutually isolated microcompartments receives a same or different cell, microtissue, cell type, cell subtype, cell donor, cell or microtissue derived from a distinct location of a subject, or cell or microtissue derived from a different subject. In some embodiments, creating the multiple experimental environments 115 includes adding multiple different co-factors or sets of co-factors to the microcompartments 110 in accordance with an experimental design for the relevant assay. In this context, the term “co-factor” refers to chemical or biological materials or species included in the microcompartments 110 in addition to cells and/or microtissues 120.

In some embodiments, the example process 500 may optionally include implanting the experimental assay array 100 into the implantation subject 107, referred to as a host animal, at operation 520. In some embodiments, the host animal receiving the implanted assay array can be provided with additional factors to interrogate the response in the different experimental microenvironments 115. For example, in some embodiments, the method further comprises administering a therapeutic agent or potential therapeutic agent to the host animal. In some embodiments, the method further comprises administering a biomolecule agent or potential biomolecule agent to the host animal. In some embodiments, the method further comprises administering growth factors, antibodies, nanoparticles, viruses, microparticles, mRNA, siRNA, CRISPR platforms, small molecules, or combination thereof, to the host animal. In some embodiments, the method further comprises administering a small molecule pharmaceutical agent or potential small molecule pharmaceutical agent to the host animal. In some embodiments, operation 520 includes observing an experimental outcome in the mutually isolated microcompartments 110 while the implanted experimental assay array is implanted. For example, operation 520 may include imaging the array in situ, as described in more detail in reference to FIG. 4.

Subsequent to implanting the implantable assay array 100, the example process 500 may optionally include recovering the implanted experimental assay array at operation 525. Operation 525 may include observing an experimental outcome in each of the mutually isolated microcompartments 110, for example, by one or more microscopy techniques including but not limited to fluorescence microscopy, confocal microscopy, light sheet microscopy, luminescence imaging, as well as biochemical assays, as would be understood by a person having ordinary skill in the art.

General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in this description and/or the claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

### EXAMPLES

This disclosure describes generation of the implantable assay array, its preparation for in vivo parallel assays, and its administration in vivo. The following paragraphs describe exemplary embodiments that are not intended to limit the scope of embodiments described in reference to FIGS. 1-5. In the examples below, the implantation subject is referred to as a “host animal,” the microcompartments are referred to as “wells,” the hydrogel formulations are referred to as “inks.” Where a “*” appears in a bar graph, the line connecting two bars of the bar graph represents a statistically significant result at P<0.05 by one-way analysis of variance (ANOVA) followed by Tukey's post hoc test.

### Example 1: Generation of the Implantable Assay Array

This Example describes an exemplary approach to generating an implantable assay array as encompassed by the present disclosure.

Initial design of the implantable assay array was assisted by use of a computer aided design (CAD) model. The initial embodiment of the array included one notched corner for orientation and identification. The design also included holes through the edges and at the corners to facilitate suturing upon implantation into the host animal.

A hydrogel substrate array printing ink was prepared by mixing PEGDA with GelMA (3.25:10 weight % 3.4 kDa PEGDA:GelMA), and adding tartrazine (1.591 mM), lithium phenyl-2,4,6-trimethylbenzoylphosphinate (17 mM), and phosphate buffered saline. The mixture was used to print the hydrogel substrate with a stereolithography 3D printer. The resulting array was washed for 3 days in PBS to remove any unreacted monomer and allow the hydrogel to swell and reach equilibrium. After 3 days, the microcompartment hydrogel formulation was prepared by mixing GelMA (3-15 wt %) with lithium phenyl-2,4,6-trimethylbenzoylphosphinate (10 mM) and cell media (PBS/EBM2). The microcompartment hydrogel formulation was pipetted into the microcompartment wells along with any cells or tissue samples. The resulting array was exposed to near-UV light for 20-180 seconds, which effected photo-crosslinking. The different weight composition of GelMA in the wells resulted in cross-linked hydrogels with different compressive moduli within the single array (i.e., 75 Pa, 100 Pa, 250 Pa, 500 Pa, 1 kPa, 3 kPa, 6 kPa, and 10 kPa), as quantified by atomic force microscopy. After photo-crosslinking, the array was complete and ready to implant.

### Example 2: Preparation and Use of Experimental Array for Parallel In vivo Assays

This Example describes production and use of experimental assay arrays prepared as described in Example 1, having 17 wells. The wells were arranged in a rectangular grid layout, with each well having a diameter of about 2 mm. The wells were seeded with 17 distinct human artificial liver “seeds” representing different combinations of human hepatocyte, endothelial cells, and fibroblasts in a biomaterial hydrogel. As described in more detail in reference to FIG. 2A. Encapsulation of the human hepatocytes within the microwells of the array had minimal or no detectable effects on hepatic cellular viability. As described in more detail in reference to FIG. 2B, which shows the results of a calcein/ethidium homodimer assay, indicating healthy aggregates of hepatocytes and fibroblasts, interspersed with randomly seeded HUVECs in the array microcompartments.

An expanded array of ˜40 individually addressable wells was fabricated and successfully used to culture hepatocytes, as described in more detail in reference to FIG. 3A and FIG. 3B. In some prototype experiments, expanded arrays were fabricated in which the stiffness of the microtissues within the array mimic the range of heterogeneous liver tissue. To accomplish variable stiffness, an array with 40 individually addressable wells was fabricated as described above. The prepolymer formulation added to the microwells was selectively varied by modifying % wt/vol of GelMA to alter crosslinking density. The variation allowed creation of hydrogels with eight different compressive moduli (i.e., 75 Pa, 100 Pa, 250 Pa, 500 Pa, 1 kPa, 3 kPa, 6 kPa, and 10 kPa). The microarray wells were seeded such that the eight different ECM conditions were seeded with five replicates in each array. Compressive stimuli were quantified by atomic force microscopy. Matrix pore size and crosslinking density were analyzed by SEM to validate any confounding variables from matrix density disparities between the ECM conditions. Finally, as in the preliminary studies, the viability of hepatocytes seeded into microarrays were confirmed using a calcein/ethidium homodimer assay

In a next assay, the role of matrix stiffness was evaluated in maintaining hepatocyte phenotype and function. To test whether that hepatocyte-specific phenotype, function, and spreading is inhibited in 3D microenvironments with increasing mechanical stiffness, rat hepatocytes were first transduced with an albumin-luciferase lentivirus. The albumin-luciferase served as a reporter for later measuring albumin promoter activity, which is a metric for hepatocyte function. The transduced rat hepatocytes were seeded in pre-polymer mixtures described above to create an array of 40 hepatic tissues with varying stiffness. After 14, 40, and 80 days in culture, bioluminescence imaging was used to assess albumin reporter activity (hepatic function) in each well of the array. The morphology and cell spreading (surrogate for cytoskeletal tension) of hepatocytes was evaluated in each condition by staining arrays with calcein, which marks living cells, and also measuring cell area. Finally, tissues were fixed and processed, and hepatic phenotype was assessed through immunostaining for hepatic markers cytokeratin-18 (Ck-18), arginase-1 (Arg-1), albumin (Alb), and alpha-1-fetoprotein (AFP).

Next, the role of matrix stiffness in liver regeneration was evaluated. To test whether that hepatocyte proliferation during liver regeneration is adversely impacted with increasing matrix stiffness, the liver microarray described above was used to test whether matrix stiffness alters hepatocyte proliferation in response to chronic liver injury. Liver microarrays, fabricated as described above, were implanted into the mesenteric fat of an immunodeficient mouse with hereditary tyrosinemia type I, a chronic liver disease, to trigger “regeneration” of the hepatocytes seeded in liver seed arrays. Five weeks after implantation, animals were pulsed with EdU and then sacrificed for histological morphometric analyses. To determine whether the liver seeds in the eight stiffness conditions differentially expanded in response to the chronic liver injury, total hepatic graft size area and hepatic maturity was quantified by immunostaining for Ck-18, Arg-1, Alb, and AFP. Further, proliferating hepatocytes were identified by double staining for Ck-18 (hepatocytes) and Ki-67, a nuclear protein associated with cellular proliferation, as well as Ck-18 and EdU, which identifies cells in the S phase of the cell cycle.

Advantageously, the advancement in understanding of the biomechanical factors mediating liver regeneration facilitates the generation of novel therapeutics aimed at enhancing liver regeneration in patients with end-stage liver diseases. Furthermore, the understanding thus derived directly informs optimal biomaterial conditions for developing engineered liver tissues as bridges or alternatives to organ transplantation. Finally, understanding how stiffness mediates liver regeneration improves fundamental understanding of conditions with dysregulated or inadequate regeneration, such as cancer, neurodegenerative disease, and cardiovascular disease.

### Example 3: Array Expansion and Additional Assays

This Example describes a scaled up array design and implementation of the assay array.

A scaled up array with 240 individually addressable wells was constructed according the general method described in Example 1. A digital light processing (DLP)-based 3D printer was used to fabricate implantable assay arrays including 240 individually addressable wells. The assay arrays included hydrogels printed using poly(ethylene glycol) diacrylate (PEGDA) and gelatin methacryloyl (GelMA), which are compatible with biological implantation. Next, the arrays were assessed for the capacity to support and promote viability of human cells. Human hepatocytes were transduced with a lentiviral vector in which luciferase is expressed under an albumin promoter. Individual wells in microarrays were seeded with human hepatocytes, endothelial cells and fibroblasts in a GelMa hydrogel. Cell viability, phenotype, and function were confirmed in array microwells using calcein/ethidium homodimer live imaging, bioluminescence imaging for spatially-localized albumin reporter activity, immunostaining for hepatic markers in clarified tissue arrays, and metrics for secreted hepatocyte products using methods established methods.

Similar to the description above in Example 2, the role of the microenvironment stiffness in human liver regenerations was assessed. HPTG was used to illuminate how mechanical stiffness of the extracellular environment and hepatocyte sourcing governs hepatocyte proliferation. Arrays were assembled in which the environmental stiffness of liver seeds in the array mimic the range of heterogeneous liver tissue across varying disease states. To vary stiffness, the hydrogel in each well were altered by modifying % wt/vol of methacrylated gelatin and/or ultraviolet exposure time. Hydrogels were created with compressive moduli ranging from 75 Pa-10 kPa, to mimic a range from sub-physiologic to cirrhotic (pathological) stiffness of liver ECM. The bioprinting methods allow spatially defining stiffness within each given liver seed in the array. Therefore, some wells were patterned to contain ‘nodules’ of differential stiffness. Wells were filled with human hepatocytes and stellate cells, both derived from either normal or cirrhotic livers, as well as endothelial cells. The arrays were implanted in injured mice and quantify hepatocyte proliferation, liver seed size and growth as above.

Additionally, an assay was conducted to test whether microliver tissues in combinatorial array grow in mice with liver injury. The combinatorial arrays containing human microliver tissue seeds were implanted onto the mesenteric fat of Fah —/— NOD, Rag1 —/—, Il2rγ null (FNRG) mice, an immune-deficient model of hereditary tyrosinemia type I. This mouse strain experiences progressive liver failure unless treated with the drug NTBC. NTBC was administered continuously to control animals or cycled on/off to induce liver damage. Immunostaining of histological tissue sections and whole clarified tissues were used to identify polarized human hepatocytes, human endothelial cells, red blood cells, and proliferating cells (EdU). Bioluminescence imaging for albumin promoter activity and circulating human hepatocyte blood products (e.g., albumin, transferrin) were used to assess microliver seed.

The implantable multi-well or multi-compartmental array facilitates testing the integrated role of many candidate factors in human liver regeneration. The biological signaling pathways that contribute to rodent liver regeneration are thought to be functionally redundant, and the relevance of these pathways to human liver regeneration is unknown. In these assays, the combinatorial regeneration array was used to assess the integrated role of rodent regenerative factors in human liver regeneration. First the individual and combinatorial roles of candidate receptors, such as MET and EGFR, were tested in human liver regeneration. The role of additional liver regeneration candidates (e.g., MET, EGFR, IL-6R, TNFR, FXR) can also be tested in every two- and three-way combination. No other system permits the scale of such exemplary studies with 56 conditions, each with four technical replicates, in a single mouse.

To test the combinatorial roles of regenerative factors, expression of MET and EGFR, which play a known combinatorial role in mouse liver regeneration, was silenced in primary human hepatocytes individually and in combination. To do this, lentiviral short-hairpin RNA, which enables stable integration of shRNA and long-term knockdown of the targeted genes can be used. Hepatocytes with silenced expression of each candidate gene(s) and control hepatocytes (scramble shRNA) were printed into different seeds of a given liver seed array. Each combinatorial condition was repeated with four technical replicates in the array. Arrays were implanted into mice with liver injury and control mice without injury. Phenotype, function, and growth of each individual microliver seed in the array were quantified as described above.

These studies established and biologically validated the multi-compartmental implantable array for efficient and scalable interrogation of human liver regeneration. Advantageously, the implantable array HPTG platform significantly accelerates comparative studies and helps advance research into treatments of devastating disease and injury to critical tissues.

### Example 4 Array Expansion and Additional Assays

This Example describes additional generation and use of prototype assay arrays.

Building physiologically complex vasculature has been a major technological challenge in tissue engineering. Microvascularization and perfusion are known to be a critical step in tissue regeneration, and the absence or even delay of this process has proven detrimental to cellular engraftment and function. It has been demonstrated that within a regenerating liver, many proangiogenic factors are upregulated, and that intrahepatic angiogenesis occurs during this phenomenon. While advances in 3D printing have led to successfully engineered vessels, 3D printing the smallest vessels on the scale of 5-10 microns remains futile. Furthermore, engineering the most optimal microenvironment for liver regeneration entails more than the ability to print exceptionally small vessels. In addition to the vascular niche, another critical microenvironmental factor is the stiffness of the extracellular matrix (ECM), which is a prominent mechanical cue that modifies cell behavior in homeostasis and disease. For example, stiffer ECMs have been shown to inhibit cellular processes such as proliferation and migration. However, the effect of matrix stiffness on both the basic biological functionality and regenerative capacity of hepatocytes is not known. This knowledge gap is due to complexities in dissecting the contribution of microenvironmental factors in liver regeneration in vivo, and to the sheer number of microenvironments that could be probed and affect hepatic function in vitro. To address this challenge, complex intravascular topologies may be printed in cell-compatible hydrogels using projection stereolithography. Using this technology, it is possible to screen photopolymerizable “ink” formulations for their angiogenic potential in bulk tissue and ability to support hepatic function both in vitro and in vivo.

Human umbilical vein endothelial cells (HUVECs) and normal human dermal fibroblasts (NHDFs) were suspended 1:1 in a total of eight formulated “inks” (3% gelMA, 5% gelMA, 10% gelMA, 15% gelMA, 10 mg/mL Fibrin, 1:1 5% gelMA:Fibrin, 1:2 5% gelMA:Fibrin, and 2:1 5% gelMA:Fibrin). Empty implantable assay arrays were pre-vascularized by filling each microwell with one of the eight experimental conditions according to a randomly generated “map.” After pre-vascularization, the arrays were implanted onto the perigonadal fat pad in Nude mice. After two weeks, the arrays were explanted and fixed. Fixed array samples were tissue-cleared using the Ce3D tissue clearing technique previously established. Cleared tissues were immunostained with antibodies against cluster for differentiation 31 (CD31) for human endothelial cells and lymphocyte antigen 76 (also known as TER-119 [clone]) for the mouse host's red blood cells and imaged using confocal microscopy (FIG. 6A and FIG. 6B).

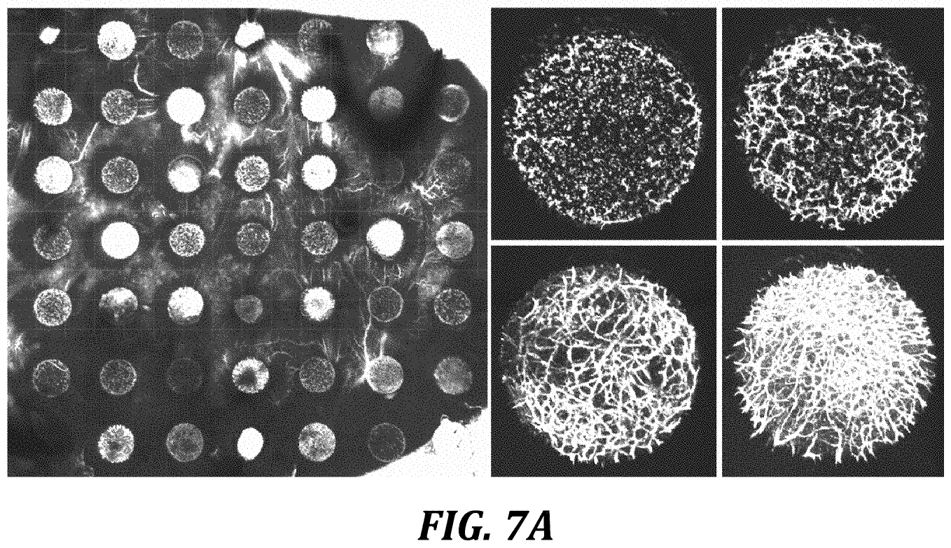

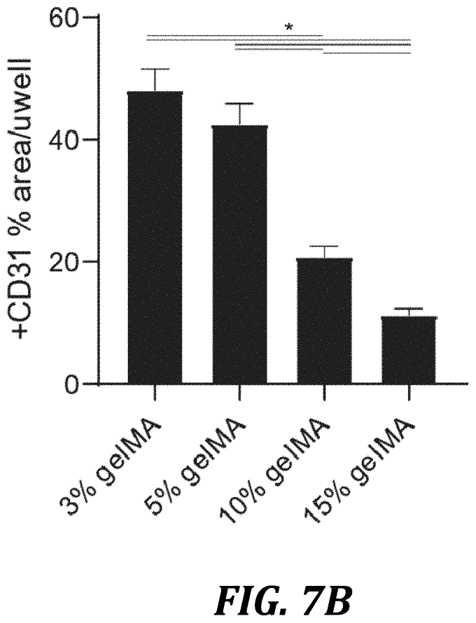

Screening eight material formulations, organized vascular networks, which were stained positive for CD31, and mouse red blood cells within these networks, indicated from positive staining of Ter-119, were preferentially evident in low GelMA protein-content material infills (See. FIG. 6A and FIG. 6B). It was observed that as the percent weight GelMA was increased, the vessel network area substantially decreased. Based on the initial large screening, four GelMA formulations were selected to investigate further with higher statistical power. The implantable assay arrays were seeded with HUVECs and NHDFs suspended in either 3%, 5%, 10% or 15% GelMA in a randomized order. The microarrays were implanted into Nude mice and analyzed as before. Organized endothelial networks formed preferentially in lower concentrations of GelMA, with the highest percentage of network area in the 3% samples (FIG. 7A and FIG. 7B). Coupled staining of CD31 and Ter-119, indicating perfusion and integration with the host network, was most notable in 3% GelMA, and for each condition percent endothelial network area was determined to be 48%, 42%, 20%, and 11% for 3%, 5%, 10%, and 15% GelMA respectively.

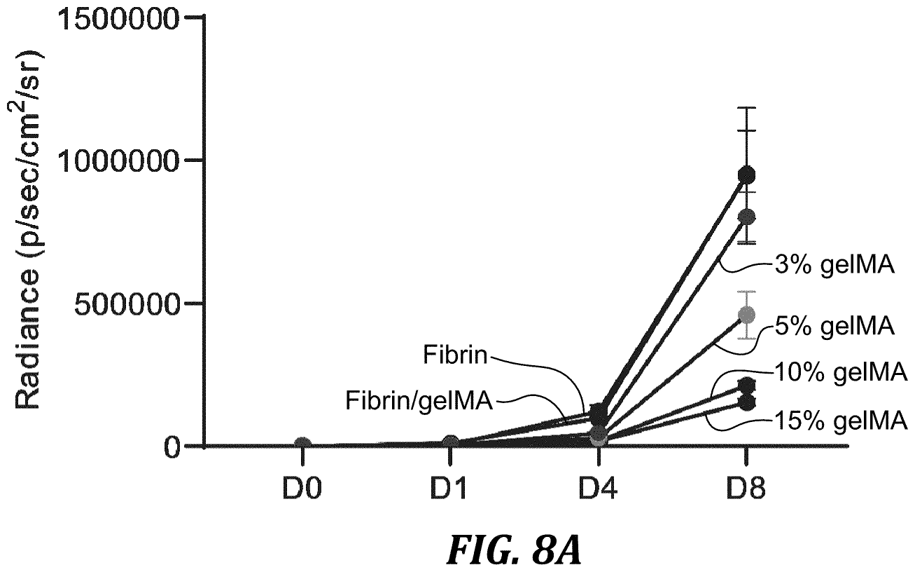

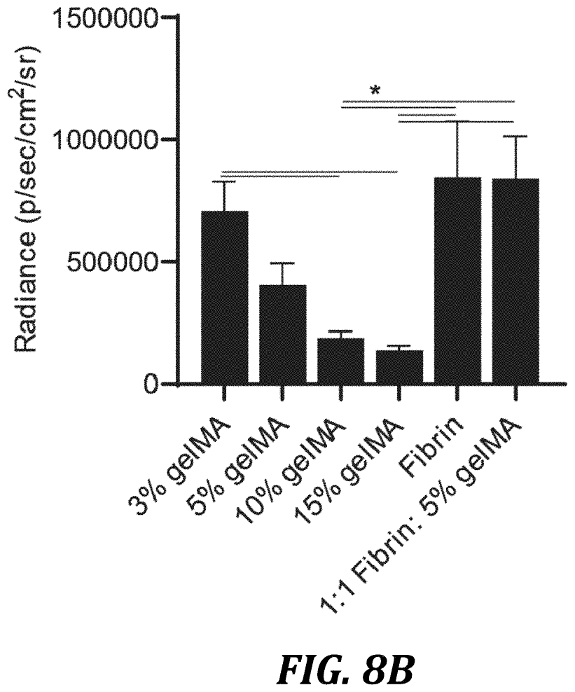

In addition to angiogenic potential, the implantable assay array was used to investigate the microenvironment effect on hepatic function in vitro. In line with results from the vascularized arrays, the previous 4 GelMA formulations were screened as well as a provisional matrix component, Fibrin, and a GelMA:Fibrin hybrid formulation. In total, these six materials were screened in one array, so that each array construct included 7-8 technical replicates of each material condition. Primary rat hepatocytes were transduced with a lentiviral vector encoding an albumin-driven luciferase reporter gene and suspended in each of the 6 conditions. Microarrays were filled according to a randomized map and cultured in vitro for 8 days. On days 0, 1, 4 and 8, luciferase radiance, an indirect metric of albumin production, was measured for each individual microwell. Lower weight percent gelMA and hybrid gelMA:Fibrin hydrogel samples preferentially supported hepatic function indicated by albumin-driven luciferase expression (See, FIG. 8A, FIG. 8B, and FIG. 8C). The increased luciferase expression over time strengthens the notion that certain microenvironments, namely 3% gelMA and gelMA:Fibrin formulations, not only support hepatic function but lead to primary hepatocyte survival in vitro, in bulk tissue long term.

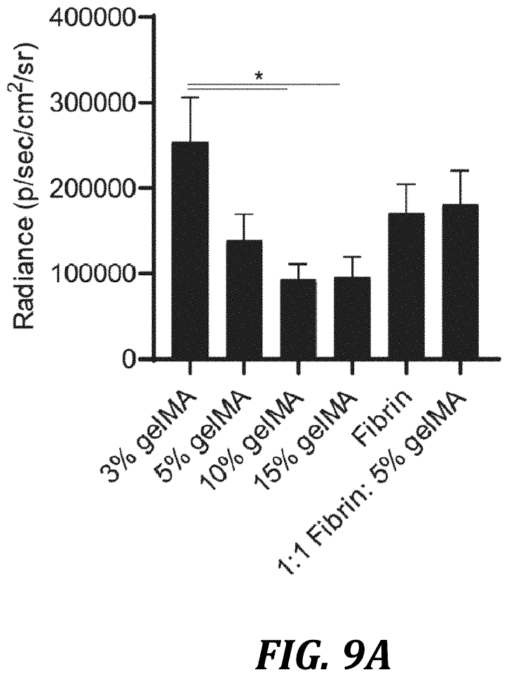

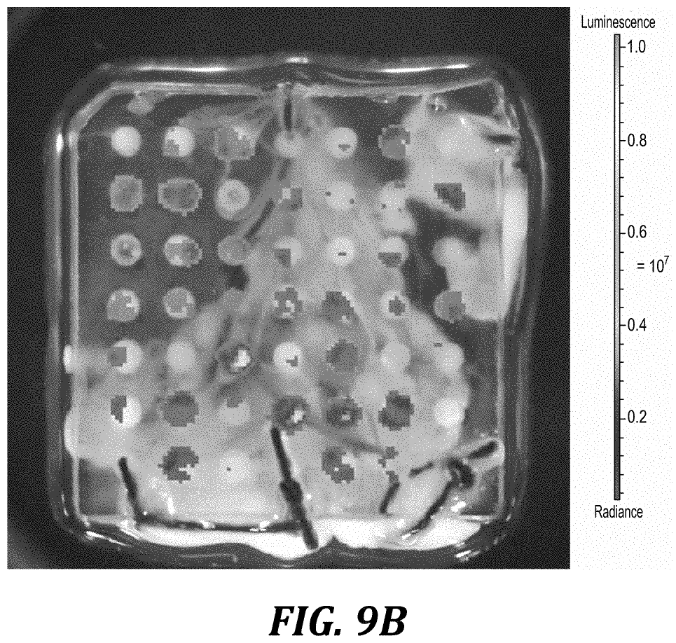

Following investigating hepatic response to the materials screen in vitro, softer inks and those containing provisional matrix components were tested to assess whether softer environments would preferentially support hepatic function in vivo. Here, arrays were prepared as before, containing the luciferase lentivirus-transduced primary rat hepatocytes suspended in the same six material inks as the in vitro screen. The hepatic microarrays were implanted in Nude mice and excised after 8 days. The explanted arrays were immediately imaged for albumin-driven luciferase expression (FIG. 9A and FIG. 9B). The results mirrored closely those of the in vitro screen. The highest albumin promoter activity, or luciferase expression, was observed in the 3% GelMA condition, followed by GelMA ink containing Fibrin. These data determined that GelMA and GelMA:Fibrin ink preferentially support hepatic viability and function in bulk tissue in vivo.

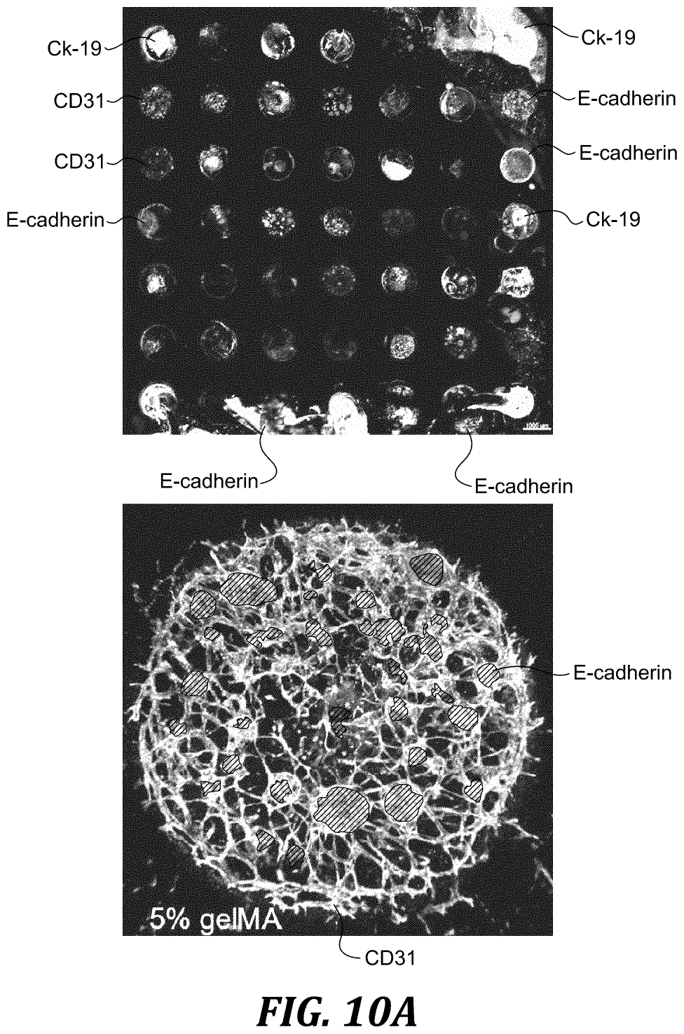

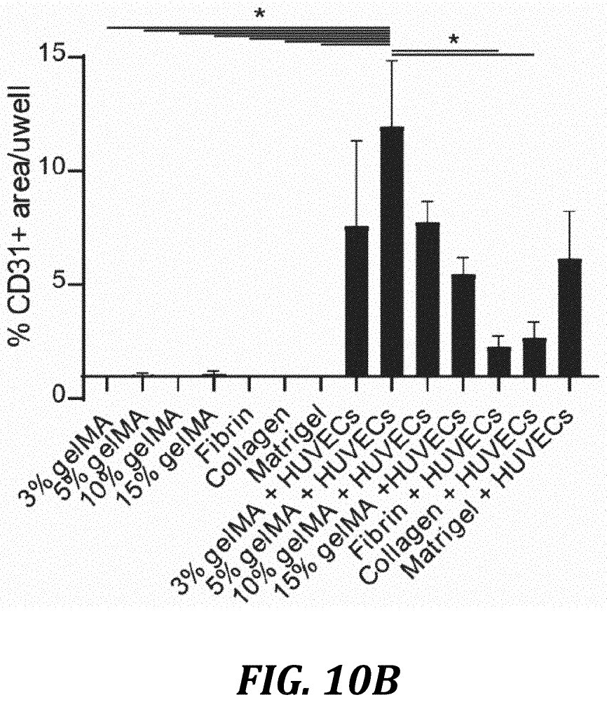

After determining that material inks can be screened for angiogenic potential and hepatic viability both in vitro and in vivo, it was sought to leverage the number of available microwells on an implantable assay array to scale up the number of experimental conditions tested in one array. Using this approach, an array map was designed that would probe hepatic engraftment as well as vascularization in a larger screen of materials concurrently. To investigate hepatic engraftment, human fetal hepatocyte organoids were seeded that were suspended in bulk in 7 different material inks (3% GelMA, 5% GelMA, 10% GelMA, 15% GelMA, Fibrin, collagen, and matrigel) either with or without HUVECs. This brings the total number of conditions screened in one microarray to 14, with 3-4 technical replicates per condition. The organoid arrays were implanted in Fah —/— NOD, Rag1 —/—, Il2rγ null (FNRG) mice, a model of liver injury. After two weeks the arrays were excised, fixed, and tissue-cleared. Clarified tissue arrays were immunostained with hepatic and human endothelial cell markers to assess organoid engraftment and function (FIG. 10A and FIG. 10B). The 5% GelMA ink preferentially supported both microvessel formation, indicated by huCD31+ networks, and hepatic engraftment, indicated by expression of a hepatic marker, E-cadherin.

Using the bioprinted screening array technology, a library of photoprintable inks were screened for capacity to support vascularization and hepatic viability and function. Various GelMA inks were screened, as well as inks incorporating traditional matrix components, including a hybrid GelMA:Fibrin material. Overall, lower concentration of GelMA lead to preferential formation of vascular networks and host perfusion as well hepatic viability and function. Here, the application of the screening array was broadened to support both in vitro and in vivo studies, each allowing for the 41 individual microwells to be successfully recovered and analyzed downstream in high throughput. These studies together were valuable for informing how to vascularize and engineer other tissues within bioprinted constructs.

The applications for the implantable screening array are not limited to the hepatic system or vascularization assays. Rather, the in vivo screening technology described herein has the capacity to be used as a tool for many other biological systems throughout the field of tissue engineering, drug screening, and personalized medicine. Many microenvironmental conditions were probed for the preferential ability to support engineered tissue survival, function, and host-vascularization in a highly parallel manner while being implanted in a smaller number of host animals, in what is termed Highly Parallel Tissue Grafting (HPTG).

While illustrative embodiments have been illustrated and described, it will be appreciated that various changes can be made therein without departing from the spirit and scope of the invention.

1. An implantable assay array, comprising:
a biocompatible substrate defining a number of mutually isolated microcompartments;
wherein the number of mutually isolated microcompartments is determined based at least in part on a size of the implantable assay array, and wherein the size of the implantable assay array is determined based at least in part on a size of an implantation subject.

2. The implantable assay array of claim 1, wherein the implantation subject is a mouse, and wherein the biocompatible substrate defines from 2 to about 500 microcompartments.

3. The implantable assay array of claim 1, wherein the implantation subject is a rat, and wherein the biocompatible substrate defines from 2 to about 50,000 microcompartments.

4. The implantable assay array of claim 1, further comprising cells or microtissue disposed in the microcompartments.

5. The implantable assay array of claim 1, wherein the cells or microtissue comprise hepatocytes, endothelial cells, stromal cells, or a combination thereof.

6. The implantable assay array of claim 1, wherein a microcompartment of the microcompartments comprises a hydrogel formulation, and wherein the hydrogel formulation comprises gelatin methacryloyl (GelMA) having a weight percent of about 1% to about 50%, poly(ethylene glycol) diacrylate (PEGDA) having a weight percent of about 1% to about 20%, fibrin, collagen, Matrigel, and cell media, or combinations thereof.

7. The implantable assay array of claim 6, wherein the hydrogel formulation is crosslinked and is characterized by a compressive modulus from about 75 Pa to about 10 kPa.

8. The implantable assay array of claim 6, wherein the hydrogel formulation comprises a nodule.

9. The implantable assay array of claim 1, wherein a microcompartment of the microcompartments comprises a biological scaffold matrix, the biological scaffold matrix comprising collagen, fibrin, decellularized extracellular matrix, silk fibroin, hyaluronic acid, hyaluronan, alginate, agarose, and methacrylated hyaluronic acid, or combinations thereof.

10. The implantable assay array of claim 1, further comprising a cover disposed on a lateral surface of the biocompatible substrate, the cover overlying the microcompartments and enclosing the crosslinked hydrogel within the microcompartment.

11. The implantable assay array of claim 9, wherein the cover transmits at least a portion of electromagnetic radiation generated by luciferase.

12. The implantable assay array of claim 1, wherein the biocompatible substrate comprises PEGDA, GelMA, collagen, fibrin, decellularized extracellular matrix, silk fibroin, hyaluronic acid, hyaluronan, alginate, agarose, and methacrylated hyaluronic acid, or combinations thereof.

13. The implantable assay array of claim 1, further comprising one or more apertures to permit suturing upon implantation into the implantation subject.

14. The implantable assay array of claim 1, further comprising a backing surface, wherein the biocompatible substrate is disposed on the backing surface.

15. The implantable assay array of claim 1, wherein the biocompatible substrate is substantially impermeable to cells, extracellular matrix, or biological molecules, such that the each microcompartment defines a biologically isolated environment.

16. The implantable assay array of claim 1, wherein each microcompartment defines an internal volume from about 0.1 μL to about 1000 μL.

17. A method of producing an implantable assay array, comprising:
incrementally depositing and photo-curing a hydrogel substrate precursor using a stereolithographic assembly, the hydrogel substrate precursor comprising a first hydrogel monomer, a photoblocker, a first photoinitiator, and cell media or biological buffer, thereby forming a biocompatible hydrogel substrate comprising a number of mutually isolated microcompartments;
disposing a hydrogel formulation, cells, microtissues, cell media, or a combination thereof, into a microcompartment of the microcompartments, the hydrogel formulation comprising a second photoinitiator and a second hydrogel monomer; and
photo-polymerizing the hydrogel formulation.

18. The method of claim 17, wherein the number of mutually isolated microcompartments is determined based at least in part on a size of the implantable assay array, and wherein the size of the implantable assay array is determined based at least in part on a size of an implantation subject to receive the implantable assay array.

19. The method of claim 17, wherein the hydrogel formulation is a first hydrogel formulation and the microcompartment is a first microcompartment, the method further comprising:
disposing a second hydrogel formulation into a second microcompartment of the microcompartments, the second hydrogel formulation being different from the first hydrogel formulation.

20. The method of claim 17, wherein the cells and/or microtissue comprise hepatocytes, endothelial cells, stromal cells, or a combination thereof.
